(19) United States
(12) Patent Application Publication
(10) Pub. No.: US 2025/0331911 A1
(43) Pub. Date: Oct. 30, 2025

(54) ENDOSCOPIC PAPILLARY LARGE SCAFFOLD DILATION

(71) Applicant: BOSTON SCIENTIFIC MEDICAL DEVICE LIMITED, Galway (IE)

(72) Inventors: Deepak Kumar Sharma, Muzaffarnagar (IN); Sharath Kumar G, Karnataka (IN)

(73) Assignee: BOSTON SCIENTIFIC MEDICAL DEVICE LIMITED, Galway (IE)

(21) Appl. No.: 19/193,440

(22) Filed: Apr. 29, 2025

Related U.S. Application Data
(60) Provisional application No. 63/640,640, filed on Apr. 30, 2024.

(51) Int. Cl. A61B 18/08; A61B 18/00

(52) U.S. Cl. CPC A61B 18/082; A61B 2018/00184; A61B 2018/00601; A61B 2018/00952

(57) ABSTRACT

A medical device includes a handle assembly and a flexible elongate member extending distally from the handle assembly. The elongate member includes a first lumen extending from the distal end of the elongate member at least partially along the elongate member toward the proximal end of the elongate member. The first lumen is configured to accept a guidewire. The elongate shaft includes a second lumen extending through at least a portion of the elongate member. A cutting wire extends through the second lumen. A distal end of the cutting wire is connected to the distal end of the elongate member, and a portion of the cutting wire extends external to the outer surface of the elongate member proximal of the distal end. An expandable scaffold is disposed at the distal portion of the elongate member. An actuator is configured to actuate the expandable scaffold between a contracted, delivery configuration and a radially expanded, deployed configuration.

### CROSS-REFERENCE TO RELATED APPLICATIONS

The application claims the benefit of U.S. Provisional Patent Application Ser. No. 63/640,640, filed on Apr. 30, 2024, the disclosure of which is incorporated herein by reference.

### TECHNICAL FIELD

The disclosure pertains to medical devices and more particularly to the field of medical devices for accessing body lumens. In particular, the present disclosure is related to medical devices, systems, and methods for targeting access to and enlarging of a body lumen opening.

### BACKGROUND

A wide variety of medical devices have been developed for medical use including, for example, devices for removal of bile duct stones (BDS's). Standard endoscopic retrograde cholangiopancreatography may not be effective in removing all types of BDS's. There is an ongoing need to provide alternative medical devices as well as alternative methods for manufacturing and using the medical devices.

### SUMMARY

This disclosure provides design, material, manufacturing method, and use alternatives for medical devices. An example medical device includes a handle assembly, a flexible elongate member extending distally from the handle assembly, the elongate member having a proximal end, a distal end, an outer surface, and a distal portion proximal to the distal end, the distal end configured to access an opening of a body lumen, and a longitudinal axis extending along a length of the elongate member, a first lumen extending from the distal end of the elongate member at least partially along the elongate member toward the proximal end of the elongate member, the first lumen configured to accept a guidewire, a second lumen extending through at least a portion of the elongate member, a cutting wire extending through the second lumen, a distal end of the cutting wire connected to the distal end of the elongate member, and a portion of the cutting wire extending external to the outer surface of the elongate member proximal of the distal end, an expandable scaffold disposed at the distal portion of the elongate member, and an actuator configured to actuate the expandable scaffold between a contracted, delivery configuration and a radially expanded, deployed configuration.

Alternatively or additionally to the embodiment above, the expandable scaffold includes a distal ring slidably disposed around the outer surface of the distal portion of the elongate member, a proximal ring fixed to the elongate member proximal of the distal ring, and a plurality of scaffolding wires extending between the proximal ring and the distal ring.

Alternatively or additionally to the embodiment above, the medical device further includes a plurality of actuating wires distally extending from the actuator to the distal ring, wherein proximal actuation of the plurality of actuating wires with the actuator moves the distal ring toward the proximal ring to expand the expandable scaffold to the radially expanded configuration.

Alternatively or additionally to the embodiment above, the plurality of actuating wires extend through the proximal ring.

Alternatively or additionally to the embodiment above, the medical device may further comprise one or more additional lumens extending through the elongate shaft, the one or more additional lumens configured to accept the plurality of actuating wires therethrough.

Alternatively or additionally to the embodiment above, the expandable scaffold includes a plurality of scaffolding wires are arranged circumferentially around the longitudinal axis of the flexible elongate member.

Alternatively or additionally to the embodiment above, the plurality of scaffolding wires are configured to deflect radially outward away from the longitudinal axis of the flexible elongate member.

Alternatively or additionally to the embodiment above, the cutting wire is configured to articulate the distal end of the flexible elongate member when the cutting wire is translated proximally through the lumen.

Alternatively or additionally to the embodiment above, wherein the handle assembly comprising finger rings actuatable to translate the cutting wire.

Alternatively or additionally to the embodiment above, the actuator is a knob slidably surrounding the elongate member.

Alternatively or additionally to the embodiment above, the knob is actuatable in a proximal direction along the elongate member to expand the expandable scaffold to the radially expanded, deployed configuration.

Alternatively or additionally to the embodiment above, the knob is configured to be locked in place when actuated to a proximal position.

Alternatively or additionally to the embodiment above, the knob is positioned between a first port and a second port on the elongate shaft.

Alternatively or additionally to the embodiment above, one of the first port and the second port is in communication with the first lumen.

Alternatively or additionally to the embodiment above, the expandable scaffold is a monolithic tubular member having a plurality of slits defining longitudinal segments therebetween.

Another example medical device includes a handle assembly, a flexible elongate member extending distally from the handle assembly, the elongate shaft having a proximal end, a distal end, an outer surface, and a distal portion proximal to the distal end, the distal end configured to access an opening of a body lumen, and a longitudinal axis extending along a length of the elongate member, a first lumen extending from the distal end of the elongate member at least partially along the elongate member toward the proximal end of the elongate member, the first lumen configured to accept a guidewire, a second lumen extending through at least a portion of the elongate member, a third lumen extending distally through the elongate member from an entry portion disposed on a proximal portion of the elongate member, the third lumen configured to accept a contrast fluid, a fourth lumen extending through at least a portion of the elongate member, a cutting wire extending through the second lumen, a distal end of the cutting wire connected to the distal end of the elongate member, and a portion of the cutting wire extending external to the outer surface of the elongate member proximal of the distal end, an expandable scaffold disposed at the distal portion of the elongate member, an actuator configured to actuate the expandable scaffold between a contracted, delivery configuration and a radially expanded, deployed configuration, and an actuation wire extending through the fourth lumen between the expandable scaffold and the actuator; wherein proximal translation of the actuation wire by the actuator causes the expandable scaffold to expand to the radially expanded, deployed configuration.

Alternatively or additionally to the embodiment above, the actuation wire extends through the proximal ring.

Another example medical device includes a flexible elongate member having a proximal end, a distal end, an outer surface, and a distal portion proximal to the distal end, the distal end configured to access an opening of a body lumen, and a longitudinal axis extending along a length of the member, a first port disposed on the elongate shaft, the first port in communication with a first lumen extending through the elongate shaft, a second port disposed on the elongate shaft, the second port in communication with a second lumen extending through the elongate shaft, a cutting wire including an exposed portion of the cutting wire extending exterior to the outer surface of the elongate member along the distal portion of the elongate shaft, the cutting wire configured to bow the distal portion of the elongate shaft into a curved shape, an expandable scaffold disposed proximal of the exposed portion of the cutting wire, and an actuator positioned between the first port and the second port, the actuator configured to translate along the elongate shaft to actuate the expandable scaffold between a contracted, delivery configuration and a radially expanded, deployed configuration.

### BRIEF DESCRIPTION OF THE DRAWINGS

The disclosure may be more completely understood in consideration of the following detailed description of various embodiments in connection with the accompanying drawings, in which:

FIG. 1 illustrates an example endoscopic sphincterotomy device;

FIG. 2 is a perspective view of the expandable scaffolding of the endoscopic sphincterotomy device of FIG. 1;

FIG. 3 illustrates a perspective view of a cross section of an elongate shaft of the endoscopic sphincterotomy device of FIG. 1;

FIG. 4 illustrates the endoscopic sphincterotomy device of FIG. 1 with the expandable scaffold in an expanded configuration.

FIGS. 5A-5E illustrate aspects of a method of using the endoscopic sphincterotomy device of FIG. 1 during a medical procedure;

FIG. 6 illustrates an alternative example expandable scaffolding for incorporation in an endoscopic sphincterotomy device such as illustrated in FIG. 1;

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “withdraw”, variants thereof, and the like, may be generally considered with respect to the positioning, direction, and/or operation of various elements relative to a user/operator/manipulator of the device, wherein “proximal” and “withdraw” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “outer extent” may be understood to mean a maximum outer dimension, “radial extent” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently-such as, but not limited to, angularly, radially, circumferentially (e.g., along an arc), etc. Additionally, the term “substantially” when used in reference to two dimensions being “substantially the same” shall generally refer to a difference of less than or equal to 5%.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The following description should be read with reference to the drawings, which are not necessarily to scale, wherein similar elements in different drawings are numbered the same. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

To help facilitate smooth and efficient entry of a guidewire and endoscopic device into/through a body lumen, medical professionals may manually rotate, oscillate, advance or “wiggle” the endoscopic device into or through the body lumen. For example, bile duct stone removal procedures require access to the bile duct through the sphincter of Oddi. Physicians in such procedures may use a sphincterotome to cannulate a body lumen (e.g. the sphincter of Oddi or the like). A sphincterotome may include an expandable portion, so that the body lumen opening may be expanded and enlarged for better access.

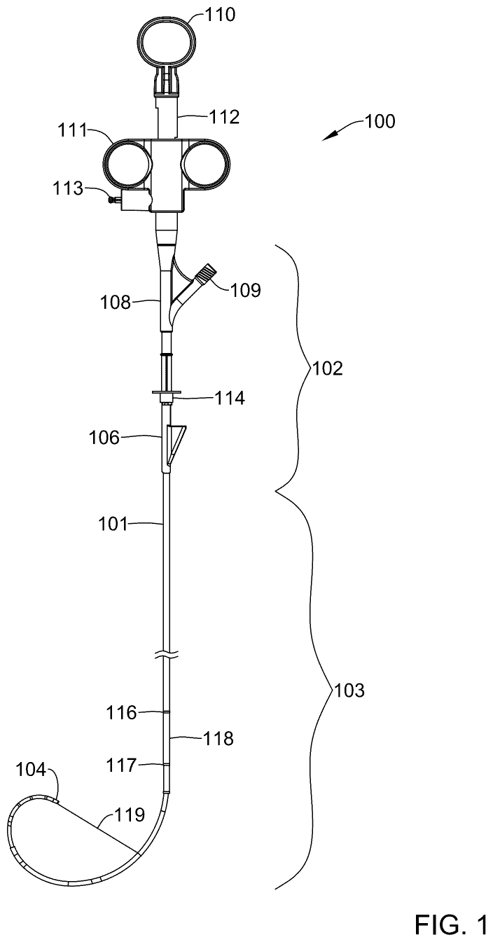

Referring to FIG. 1, a medical device for use in endoscopic body lumen access, such as retrograde cholangiopancreatography (ERCP) procedures. The medical device may be considered an endoscopic sphincterotomy device. The medical device includes a handle assembly, including a handle body 112, and a catheter apparatus 100. The handle body 112 has a proximal grip ring 110 which is stationary. The grip ring 110 and handle body 112 may be a single monolithic element, or may be two distinct pieces that have been attached through any known method. The handle assembly also includes an actuatable member, such as slidable finger rings 111, disposed around the handle body 112, configured to be actuated relative to the handle body 112. For example, the finger rings 111 may be located distal of the grip ring 110, such that the finger rings 111 are longitudinally actuatable relative to the grip ring 110. The figure rings 111 may be slidable until they reach a stopper at the distal end of the handle body, and a connector 113 may be attached to the finger rings 111 or the handle body 112. It should be understood that the connector 113 may be attached to any element of the handle, or may be disposed along the catheter apparatus 100. The connector 113 may be coupleable to an energy source, to provide electrical energy to the cutting wire 119, as will be described further herein.

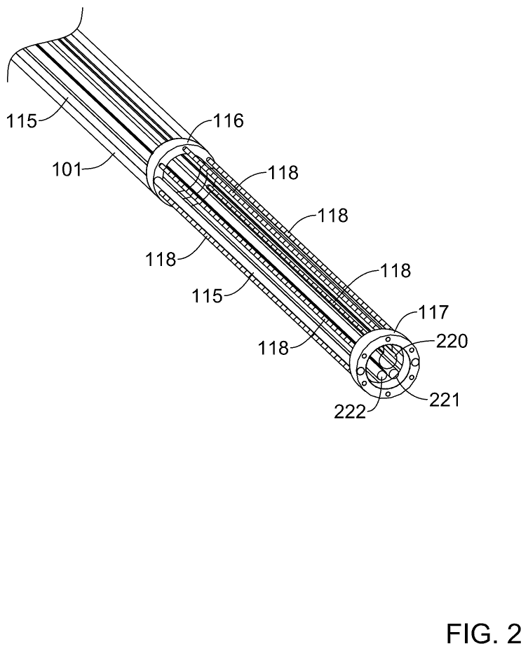

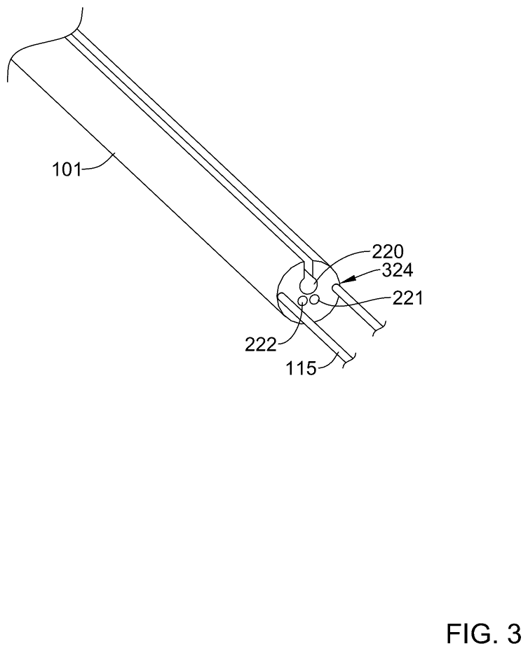

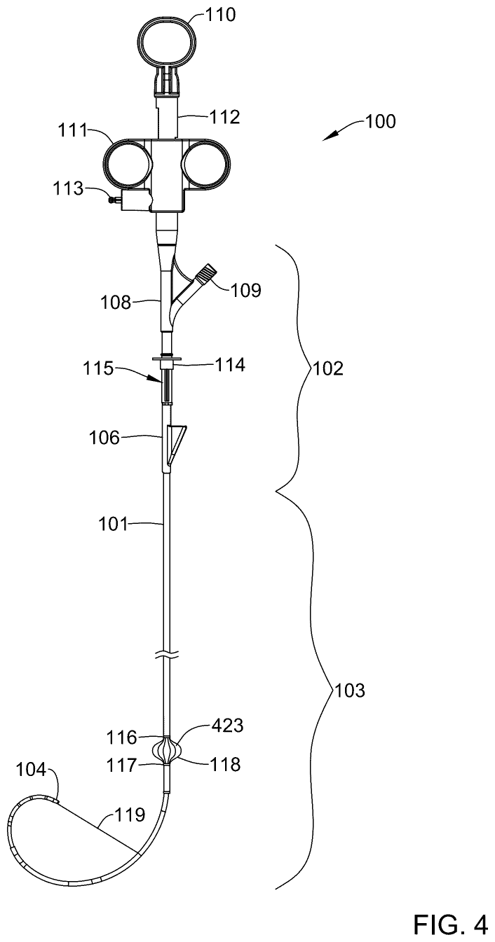

The distal end of the handle body 112 may be attached (e.g., fixedly attached) to a proximal portion 102 of the catheter apparatus. They handle body 112 and catheter 100 may be attached through any known methods (fitting, adhesives and the like). The catheter apparatus 100 includes a proximal portion 102 extending distally from a distal end of the handle assembly, and a distal portion 103 extending proximally from the distal end 104 of the catheter shaft 101. The proximal portion 102 may include entry ports 106 and 108 which may give a user access into lumens of the catheter shaft 101 of the catheter apparatus 100. The entry ports may also include a fitting 109 disposed on one, both, or neither of the entry ports. One of ordinary skill in the art would understand that this specific port configuration is given as an example and is not meant to be limiting, various other configurations would be apparent to one of ordinary skill in the art. The proximal portion 102 also includes an actuator, such as a slidable knob 114, operatively connected to scaffold actuating wires 115 (shown in FIGS. 2 and 3). The actuator, e.g., knob 114 is slidable between a distal position as shown in FIG. 1 and a proximal position as shown in FIG. 4.

The distal portion 103 of the catheter apparatus 100 includes the catheter shaft 101, proximal and distal scaffolding rings 116, 117, expandable scaffold 118, and catheter cutting wire 119. In some instances, the distal portion 103 of the catheter apparatus 101 may have a smaller diameter than the proximal portion 102, to increase the flexibility of the distal portion 103. The catheter cutting wire 119 extends through a lumen of the catheter apparatus 100, before exiting and reentering the distal portion 103 of the catheter shaft 101, such that a portion of the cutting wire 119 extends out of, extends along an exterior of the distal portion 103 of the catheter shaft 101, and is visible exterior of the catheter shaft 101. Said differently, part of the cutting wire 119 may be located exterior of the catheter shaft 101 at all times. The proximal end of the catheter cutting wire 119 is operatively connected to the slidable finger rings 111, and a distal end of the catheter cutting wire 119 may be fixed at a distal end 104 to the catheter shaft 101. Proximal actuation of the slidable finger rings 111, e.g., in a proximal direction toward the grip ring 110, may cause the distal end region of the catheter shaft 101 to bow in an arcuate shape, with the cutting wire 119 extending therebetween. The handle body 112 may include a pulley system or other similar mechanism to affect the cutting wire 119. Actuation of the finger rings 111, or other actuator, in the proximal or distal direction, correlates to actuation of the distal tip 104 of the catheter shaft 101, causing the catheter shaft 101 to have a curved or bowed shape. Manipulation of the catheter shaft 101 may be used to improve control, efficiency, and ease of access into body lumens, as well as assist in treatment (e.g. cauterizing tissue, removal of bile duct stones or the like).

The expandable scaffold 118 is arranged on the catheter shaft 101 proximal of the catheter cutting wire 119. The expandable scaffold 118 may be positioned between the distal scaffold ring 117 and the proximal scaffold ring 116. In some instances, at least one of the proximal and distal scaffold rings 116, 117 may be slidable relative to the catheter shaft 101 such that moving the proximal and distal scaffold rings 116, 117 closer together expands the expandable scaffold 118 positioned therebetween. For example, the distal scaffolding ring 117 may slidable around the catheter shaft 101 while the proximal scaffolding ring 116 may be fixed to the catheter shaft 101. In such an instance, the distal scaffolding ring 117 may be connected to a distal end of the flexible scaffold 118 and to a distal end of the scaffold actuating wires 115. The flexible scaffold 118 may extend proximally from the distal scaffolding ring 117 to the proximal scaffolding ring 116, which is fixed to the catheter shaft 101. The proximal and distal rings 116, 117, as well as the scaffolding wires 118 may be disposed on the exterior of the catheter shaft 101, such that the catheter shaft 101 extends through each of the proximal scaffolding ring 116, the expandable scaffold 118, and the distal scaffolding ring 117. However, in some embodiments, there may be a break in the catheter shaft 101 between the proximal 116 and distal 117 rings.

As shown in FIG. 2, the expandable scaffold 118 may include one or more, or a plurality of scaffold wires extending between the proximal scaffolding ring 116 and the distal scaffolding ring 117, with a proximal end of each of the scaffold wires fixed to the proximal scaffolding ring 116 and a distal end of each of the scaffold wires fixed to the distal scaffolding ring 117. The scaffold wires may be sufficiently flexible such that the scaffold wires extend generally parallel to the longitudinal axis of the catheter shaft 101 in a radially contracted, delivery configuration (shown in FIG. 2), while a middle region of the scaffold wires are deflected or bowed radially outward away from the longitudinal axis of the catheter shaft 101 in an expanded, deployed configuration (shown in FIG. 4).

As is shown in FIGS. 2 and 3, there may be lumens 220, 221, 222 extending along the length of the catheter shaft 101, which are continuous through the entire shaft including the scaffolding portion. The scaffold actuating wires 115 extend proximally from the distal scaffolding ring 117 to the actuator (e.g., knob 114), and are slidable through lumens 324 (FIG. 3) of the proximal scaffolding ring 116. A proximal portion of the scaffold actuating wires 115 is connected to the knob 114. Movement of the knob 114, or other actuator, in the proximal direction, correlates with proximal longitudinal movement of the scaffold actuating wires 115 and distal scaffolding ring 117. In other words, proximal translation of the scaffold actuating wires 115 causes the distal scaffolding ring 117 to translate proximally toward the proximal scaffolding ring 116, thereby expanding the expandable scaffold 118. As the distal ring 117 is pulled in the proximal direction, the distance between the proximal ring 116 and the distal ring 117 decreases, causing radial expansion of the scaffold wires of the expandable scaffold 118. There may be any number of scaffolding wires extending between the scaffolding rings 116 and 117. In some instances, the expandable scaffold 118 may also include an expandable polymeric sheath 423 extending between the scaffolding rings 116 and 117 (FIG. 4) and covering the scaffolding wires, if desired. The sheath 423 may have the added benefit of providing more even pressure to the surrounding tissue as the expandable scaffold 118 expands, and preventing the scaffolding wires from catching on anything while in the delivery configuration. The sheath 423 may be attached to the rings 116, 117, the scaffolding wires of the expandable scaffold 118, any combination thereof or the like. The expandable sheath 423 may be made of any flexible material known in the art.

FIG. 2 also illustrates a more detailed view of the scaffolding portion of the catheter shaft 101 as discussed in previous embodiments. The proximal and distal rings 116, 117 surround the catheter shaft 101. Any number of scaffolding wires forming the expandable scaffold 118 may extend between the two rings 116, 117, and the wires 118 may be attached to the rings through any known method e.g. adhesives, crimping, through a lumen, or the like. The scaffolding wires are shown extending exterior of the catheter shaft 101, and are unconstrained by the catheter shaft 101, allowing for radial expansion of the wires of the expandable scaffold 118. The proximal ring 116 is fixed to the catheter shaft 101, and has lumens configured to receive the scaffolding actuating wires 115 therethrough. The scaffolding actuating wires 115 extend proximally from the distal ring 117, through the proximal ring 116, through one or more lumens of the catheter shaft 101 proximal of the expandable scaffold 118, and to the knob 114 (FIG. 1).

Referring to FIG. 3, a cross section of the distal portion 103 of the catheter shaft 101 is shown. The interior of the catheter shaft 101 includes a guidewire exchange channel 220, a cautery wire lumen 221, and a contrast lumen 222. The guidewire exchange channel 220 may be configured to receive a guidewire through an entire length of the channel 220, or a portion of the channel 220 extending to the distal end of the catheter shaft 101. The guidewire channel 220 may be in communication with one of the entry ports 106 or 108, such as the port 106 to provide access for a guidewire to enter the guidewire exchange channel 220. The cautery wire lumen 221 may be configured to receive an electrically conductive wire (not shown) through the length of the channel or a portion of the channel extending to the distal end of the catheter shaft 101. The cautery wire may receive electrical energy from the connector 113 shown in FIG. 1. The cautery wire an extending of the cutting wire 119, or may otherwise be electrically connected to the cutting wire 119 to provide electrical energy to the cutting wire 119. The contrast lumen 222 may be configured to receive a contrast fluid from one of the entry ports 106 or 108, such as the port 108. The lumens 220, 221 and 222 may be configured for many uses e.g. receiving a guidewire, articulating wire, fluid, any combination thereof, or the like. The size and position of the lumens 220, 221, 222 are not intended to be limiting and it should be understood that the lumens may be sized and shaped to accommodate a number of wires, fluids, medical devices, any combination thereof or the like.

Referring now to FIG. 4, which shows the medical device of FIG. 1 with the expandable scaffold 118 in the expanded configuration. As noted above, the expandable scaffold 118 may be expanded by actuating the actuator in a proximal direction, e.g., shown with the knob 114 withdrawn proximally. The knob 114 may include a lock to secure the knob in the proximal position, and thus retain the expandable scaffold 118 in the expanded configuration. The knob 114 may be locked in the proximal position by any known means e.g. magnet, switch, button, twist lock or the like. As the knob 114 is pulled in a proximal direction, the actuating wires 115 and distal ring 117 are pulled in a proximal direction. As the distance between the distal ring 117 and the proximal ring 116 decreases, the scaffolding wires of the expandable scaffold 118 expand radially. Said differently, proximal movement of the knob 114 causes radial expansion of the scaffolding wires of the expandable scaffold 118. The wires of the expandable scaffold 118 may be configured to expand uniformly, or may have varying shapes and directions of expansion. The distance between the proximal ring 116 and distal ring 117, as well as the distance that the knob 114 is able to be actuated may all vary to allow for a user to expand the scaffolding as much, or as little as desired. In other words, the knob 114 may be actuated proximally to any one of a plurality of positions to cause the expandable scaffold 118 to correspondingly expand to any one of a plurality of radial extents. As the wires of the expandable scaffold 118 expand, the expandable sheath 423 (if provided) also expands. In some instances, the sheath 423 may be configured to adhere tightly to the wires of the expandable scaffold 118, having small dips between the wires of the expandable scaffold 118 where the sheath 423 is not directed supported, forming a fluted structure. In some embodiments, the sheath 423 may be configured to take on a substantially round or ovular shape as the wires of the expandable scaffold 118 expand. The sheath 423 may be configured to expand and contract with the expandable scaffold 118. The amount of expansion that the scaffolding 118 undergoes may be decided by a physician during a procedure. The amount of expansion desired may depend on the unique anatomy of a patient and/or the type of procedure e.g. removing a bile stone whole, in pieces etc. The wires of the expandable scaffold 118 may be any length, width, and flexibility allowing for the size and shape of the expanded scaffold 118 to suit any unique anatomy and type of procedure. The wires of the expandable scaffold 118 may also have a coating, if desired.

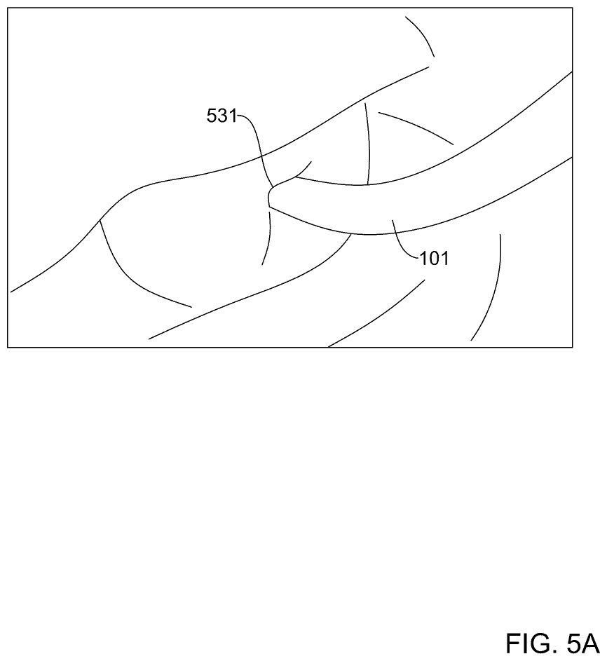

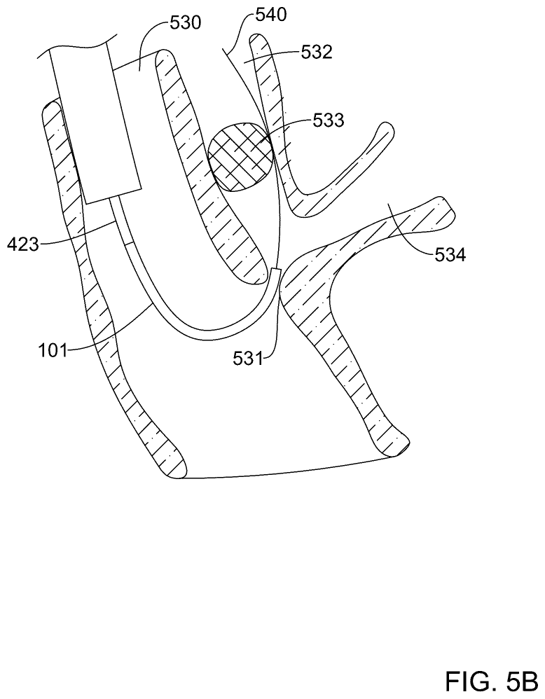

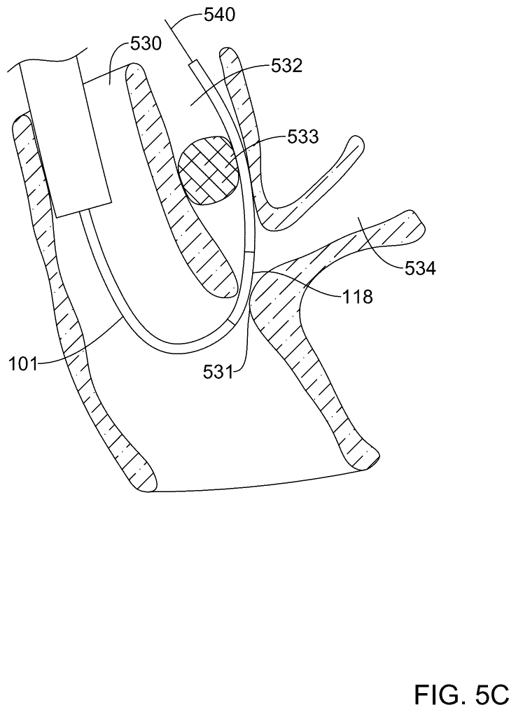

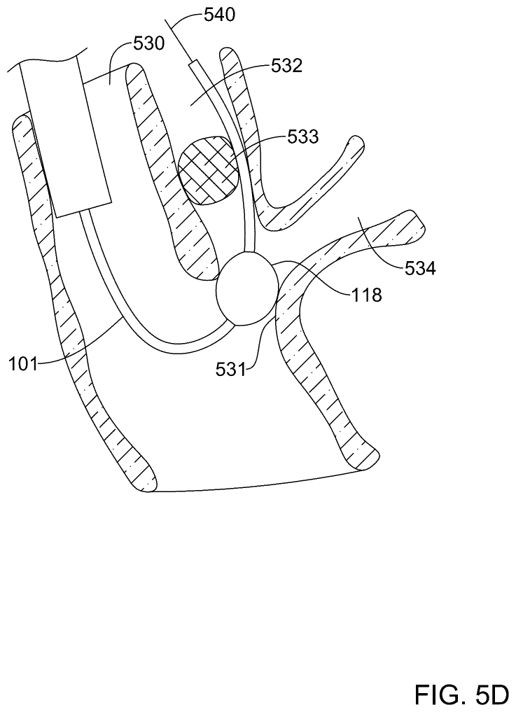

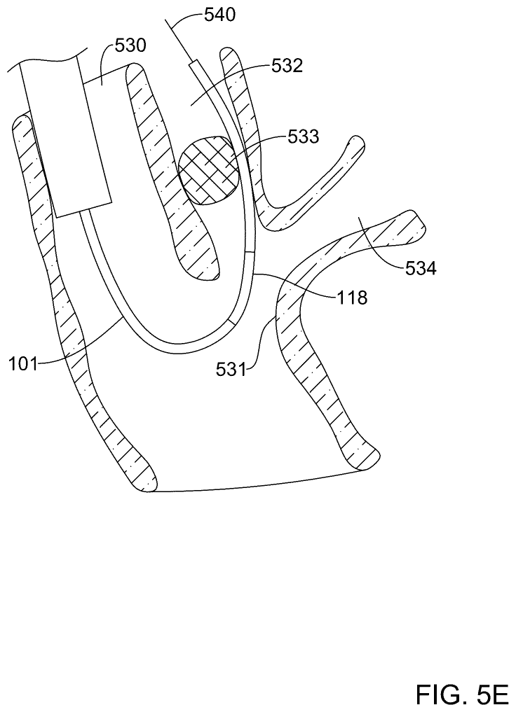

FIG. 5A shows a perspective view of the sphincter of Oddi 531 with the catheter shaft 101 inserted into it. It should be known that any similar body lumen or sphincter may be substituted for the Sphincter of Oddi 531 discussed herein. As can be seen in FIG. 5B, the sphincter of Oddi 531 leads to the bile duct 532 and the pancreatic duct 534. The device 100 (FIG. 1) is advanced through a body lumen 530 to reach the sphincter of Oddi 531. A guidewire 540 may be advanced through the sphincter of Oddi 531, bypassing the pancreatic duct 534 to reach the bile duct 532. The guidewire 540 may be maneuvered past or around a bile duct stone 533 located within the bile duct 532. The catheter shaft 101 may follow the path of the guidewire 540 and be advanced through the sphincter 531 into the bile duct 532. Once positioned across the sphincter 531, the catheter may be actuated to deflect the distal end region of the catheter to cut the sphincter 531 with the cutting wire 119 (not expressly shown). Thereafter, as shown in FIG. 5C, the catheter 101 may be advanced until the expandable scaffolding 118 reaches the sphincter of Oddi 531 in its delivery configuration. As shown in FIG. 5D, the expandable scaffolding 118 may be expanded to its expanded configuration, effectively dilating the sphincter of Oddi 531. The sphincter 531 may be dilated any amount desired with the expandable scaffolding 118, and in some cases may be dependent on the size of the bile stone 532, the type of removal procedure being used, amount of visualization required, and/or the unique anatomy of the patient. After expansion, the expandable scaffolding 118 may return to its collapsed delivery configuration, leaving the sphincter 531 expanded. The sphincter 531, being expanded, may allow increased visualization of the bile duct, and easier removal of stones in difficult cases e.g. periampullary diverticulum, altered anatomy after gastric bypass, the shape/size of bile duct stone is difficult to determine for mechanical removal and extraction, or the like. The catheter shaft 101 may include fluoroscopic portions along its length, to allow for visualization of the position of the device 100 during use.

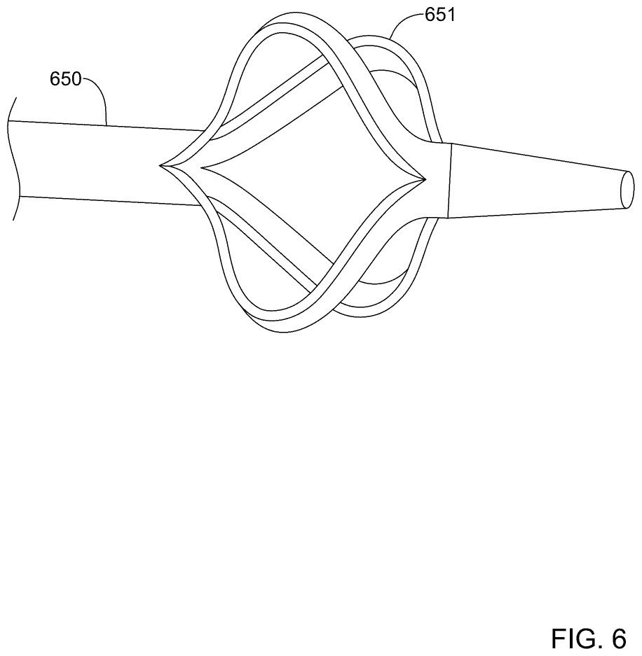

As shown in FIG. 6, in an alternative configuration, the expandable scaffolding structure may be formed of a monolithic tubular sheath 650 having slits between adjacent longitudinal segments 651 of the monolithic tubular sheath 650 which allow for radial expansion of the longitudinal segments 651 of the sheath 650. The sheath 650 may replace the actuation wires 115 and rings 116, 117 of other embodiments. The sheath 650 may have any number of slits, an any number of longitudinal segments 651 along a length of the sheath 650. Thus, the longitudinal segments 651 may be integrally formed from the tubular member forming the sheath 650. In some embodiments the expandable scaffold may be made of a mesh of wires, such as a braided mesh of wires.

It will be understood that the dimensions described in association with the above figures are illustrative only, and that other dimensions are contemplated. The materials that can be used for the various components of the medical device and the various elements thereof disclosed herein may include those commonly associated with medical devices. For simplicity purposes, the following discussion makes reference to the medical device (and variations, systems or components disclosed herein). However, this is not intended to limit the devices and methods described herein, as the discussion may be applied to other elements, members, components, or devices disclosed herein.

In some embodiments, the medical device (and variations, systems or components thereof disclosed herein) may be made from a metal, metal alloy, ceramics, zirconia, polymer (some examples of which are disclosed below), a metal-polymer composite, combinations thereof, and the like, or other suitable material. Some examples of suitable metals and metal alloys include stainless steel, such as 444V, 444L, and 314LV stainless steel; mild steel; nickel-titanium alloy such as linear-elastic and/or super-clastic nitinol; cobalt chromium alloys, titanium and its alloys, alumina, metals with diamond-like coatings (DLC) or titanium nitride coatings, other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276R, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R44035 such as MP35-NR and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R44003 such as ELGILOY®, PHYNOX®, and the like); platinum enriched stainless steel; titanium; platinum; palladium; gold; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “super-elastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear than the super elastic plateau and/or flag region that may be seen with super elastic nitinol. Thus, for the purposes of this disclosure linear elastic and/or non-super-elastic nitinol may also be termed “substantially” linear elastic and/or non-super-clastic nitinol.

In at least some embodiments, portions or all of the medical device (and variations, systems or components thereof disclosed herein) may also be doped with, made of, or otherwise include a radiopaque material. Radiopaque materials are understood to be materials capable of producing a relatively bright image on a fluoroscopy screen or another imaging technique during a medical procedure. This relatively bright image aids a user in determining the location of the medical device (and variations, systems or components thereof disclosed herein). Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the medical device (and variations, systems or components thereof disclosed herein) to achieve the same result.

In some embodiments, the medical device (and variations, systems or components thereof disclosed herein) and/or portions thereof, may be made from or include a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, polyurethane silicone copolymers (for example, Elast-Eon® from AorTech Biomaterials or ChronoSil® from AdvanSource Biomaterials), biocompatible polymers, other suitable materials, or mixtures, combinations, copolymers thereof, polymer/metal composites, and the like. In some embodiments, the sheath can be blended with a liquid crystal polymer (LCP). For example, the mixture can contain up to about 6 percent LCP.

In some embodiments, the medical device (and variations, systems or components thereof disclosed herein) may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vascoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A medical device, comprising:
a handle assembly;
a flexible elongate member extending distally from the handle assembly, the elongate member having a proximal end, a distal end, an outer surface, and a distal portion proximal to the distal end, the distal end configured to access an opening of a body lumen, and a longitudinal axis extending along a length of the elongate member;
a first lumen extending from the distal end of the elongate member at least partially along the elongate member toward the proximal end of the elongate member, the first lumen configured to accept a guidewire;
a second lumen extending through at least a portion of the elongate member;
a cutting wire extending through the second lumen, a distal end of the cutting wire connected to the distal end of the elongate member, and a portion of the cutting wire extending external to the outer surface of the elongate member proximal of the distal end;
an expandable scaffold disposed at the distal portion of the elongate member; and
an actuator configured to actuate the expandable scaffold between a contracted, delivery configuration and a radially expanded, deployed configuration.

2. The medical device of claim 1, wherein the expandable scaffold includes:
a distal ring slidably disposed around the outer surface of the distal portion of the elongate member;
a proximal ring fixed to the elongate member proximal of the distal ring; and
a plurality of scaffolding wires extending between the proximal ring and the distal ring.

3. The medical device of claim 2, further comprising
a plurality of actuating wires distally extending from the actuator to the distal ring,
wherein proximal actuation of the plurality of actuating wires with the actuator moves the distal ring toward the proximal ring to expand the expandable scaffold to the radially expanded configuration.

4. The medical device of claim 3, wherein the plurality of actuating wires extends through the proximal ring.

5. The medical device of claim 4, further comprising one or more additional lumens extending through the elongate shaft, the one or more additional lumens configured to accept the plurality of actuating wires therethrough.

6. The medical device of claim 1, wherein the expandable scaffold includes a plurality of scaffolding wires are arranged circumferentially around the longitudinal axis of the flexible elongate member.

7. The medical device of claim 6, wherein the plurality of scaffolding wires is configured to deflect radially outward away from the longitudinal axis of the flexible elongate member.

8. The medical device of claim 1, wherein the cutting wire is configured to articulate the distal end of the flexible elongate member when the cutting wire is translated proximally through the lumen.

9. The medical device of claim 1, wherein the handle assembly comprises finger rings actuatable to translate the cutting wire.

10. The medical device of claim 1, wherein the actuator is a knob slidably surrounding the elongate member.

11. The medical device of claim 10, wherein the knob is actuatable in a proximal direction along the elongate member to expand the expandable scaffold to the radially expanded, deployed configuration.

12. The medical device of claim 11, wherein the knob is configured to be locked in place when actuated to a proximal position.

13. The medical device of claim 11, wherein the knob is positioned between a first port and a second port on the elongate shaft.

14. The medical device of claim 13, wherein one of the first port and the second port is in communication with the first lumen.

15. The medical device of claim 1, wherein the expandable scaffold is a monolithic tubular member having a plurality of slits defining longitudinal segments therebetween.

16. A medical device, comprising:
a handle assembly;
a flexible elongate member extending distally from the handle assembly, the elongate shaft having a proximal end, a distal end, an outer surface, and a distal portion proximal to the distal end, the distal end configured to access an opening of a body lumen, and a longitudinal axis extending along a length of the elongate member;
a first lumen extending from the distal end of the elongate member at least partially along the elongate member toward the proximal end of the elongate member, the first lumen configured to accept a guidewire;
a second lumen extending through at least a portion of the elongate member;
a third lumen extending distally through the elongate member from an entry portion disposed on a proximal portion of the elongate member, the third lumen configured to accept a contrast fluid;
a fourth lumen extending through at least a portion of the elongate member;
a cutting wire extending through the second lumen, a distal end of the cutting wire connected to the distal end of the elongate member, and a portion of the cutting wire extending external to the outer surface of the elongate member proximal of the distal end;
an expandable scaffold disposed at the distal portion of the elongate member;
an actuator configured to actuate the expandable scaffold between a contracted, delivery configuration and a radially expanded, deployed configuration; and
an actuation wire extending through the fourth lumen between the expandable scaffold and the actuator; wherein proximal translation of the actuation wire by the actuator causes the expandable scaffold to expand to the radially expanded, deployed configuration.

17. The medical device of claim 16, wherein the expandable scaffold includes:
a distal ring slideably disposed around the outer surface of the distal portion of the elongate member, the distal ring coupled to a distal end of the actuation wire;
a proximal ring affixed to the elongate member proximal of the distal ring; and
a plurality of scaffolding wires extending between the proximal ring and the distal ring.

18. The medical device of claim 17, wherein the actuation wire extends through the proximal ring.

19. The medical device of claim 17, wherein the actuator includes a knob that is actuatable in a proximal direction to expand the expandable scaffold to the radially expanded, deployed configuration.

20. A medical device, comprising:
a flexible elongate member having a proximal end, a distal end, an outer surface, and a distal portion proximal to the distal end, the distal end configured to access an opening of a body lumen, and a longitudinal axis extending along a length of the member;
a first port disposed on the elongate shaft, the first port in communication with a first lumen extending through the elongate shaft;
a second port disposed on the elongate shaft, the second port in communication with a second lumen extending through the elongate shaft;
a cutting wire including an exposed portion of the cutting wire extending exterior to the outer surface of the elongate member along the distal portion of the elongate shaft, the cutting wire configured to bow the distal portion of the elongate shaft into a curved shape;
an expandable scaffold disposed proximal of the exposed portion of the cutting wire; and
an actuator positioned between the first port and the second port, the actuator configured to translate along the elongate shaft to actuate the expandable scaffold between a contracted, delivery configuration and a radially expanded, deployed configuration.
